(19) United States
(12) Patent Application Publication
(10) Pub. No.: US 2007/0207552 A1
(43) Pub. Date: Sep. 6, 2007

(54) Device for investigating chemical interactions and process utilizing such device

(75) Inventors: Johannes Gijsbertus Antonius Terlingen, Landgraaf (NL); Gerardus Henricus Maria Engbers, Oldenzaal (NL)

Correspondence Address: THE WEBB LAW FIRM, P.C., 700 KOPPERS BUILDING, 436 SEVENTH AVENUE, PITTSBURGH, PA, 15219, US

(73) Assignee: Holland Biomaterials Group B.V., NB ENSCHEDE (NL)

(21) Appl. No.: 11/699,927

(22) Filed: Jan. 30, 2007

Related U.S. Application Data
(63) Continuation of application No. 09/762,779, filed on Jul. 3, 2001, now abandoned.

(30) Foreign Application Priority Data
Aug. 14, 1998 (NL) NL 1009871

(51) Int. Cl. G01N 33/00; B05D 1/02

(52) U.S. Cl. 436/183; 427/447; 73/866

(57) ABSTRACT

The invention relates to a device for investigating reactions between interactive species, said device comprising: one or more plasma deposited layers, which layers comprise one or more first pre-selected functional group species, which functional group species are interactible with a pre-selectable second species.

The present invention relates to a device for investigating reactions between interactive chemical and/or biological species, to a process for providing such a device, and to a process for investigating chemical and/or biological interactions, for example biomolecular interactions, utilizing such a device.

Under chemical and/or biological interactions is also understood chemical and/or biological reactions.

Interactions of specific compounds with solid surfaces play a crucial role in chemical and biological phenomena and areas including analysis techniques such as RIA's, ELISA's.

For investigating and sensing surface interactions a ‘sensitive’ surface is required.

To study real time surface interactions several techniques are available such as ellipsometry, reflectometry and surface plasmon resonance spectroscopy (SPR). These techniques have in common that they use the reflectance of light, generated by a laser, to analyze the growth or desintegration of a layer of for instance biological molecules at a surface.

For these techniques, a reflecting surface is necessary. In the case of SPR, a surface comprising a free electron metal for example gold is most frequently used.

In order to utilize this technique for investigating other interactions, besides the interaction of (bio)molecules with free electron metal surfaces, the free electron surfaces have been modified, for instance, by the adsorption of bio-molecules such as proteins and the coating thereof with polymeric layers in a solvent cast or spin coat procedures.

Methods have also been developed to provide gold surfaces with specific chemical groups for the immobilization of proteins, which surfaces are subsequently utilized for studying the interactions with other (biological) substances such as antibody-antigen interactions.

Methods for generating SPR sensor surfaces include arranging an organic surface onto a gold layer by means of a wet chemistry procedure such as solvent casting or spin coating before carrying out a plasma etching procedure.

A further method includes adsorption of a chemical functional surfactant, by means of a wet chemistry procedure, on the surface to be modified and the subsequent immobilization of the surfactant by a plasma such as an argon plasma, so called plasma immobilization.

Disadvantages of these known techniques include the lack of stability of the functional surface layers.

An object of the present invention is to provide an improved device for investigating the reactions between interactive chemical species.

According to a first aspect of the present invention there is provided a device according to any of the claims 1 to 8.

The device according to the present invention provides a good attachment of the plasma deposited layer, a good stability thereof and a device exhibiting good sensitivity, whereby the substrate is provided with a functional layer, the functionality of which can be provided by groups such as amine, carboxylic acid, hydroxyl, acid chloride, isocyanate, aldehyde, anhydride, epoxide, and thiol groups for example.

According to a second aspect of the present invention, there is provided a process according to any of the claims 9 to 19 for providing the device according to the present invention.

Since a functional group layer is plasma deposited, control over the deposition thereof—can be accurately carried out, whereby very thin layers can be deposited thus providing very sensitive devices, without the need for firstly arranging an organic layer by wet chemical methods on the substrate before any further investigation can be carried out.

The process according to the present invention provides a good controllability.

In contrast to processes for providing sensor devices, wherein layers are arranged on a substract by wet chemical processes which are often time consuming, difficult to carry out, and often result in undesirably thick layers exhibiting a subsequent lack of sensitivity if a great deal of care in not applied, the process according to the present invention is extremely flexible to work and easy to effect and offers a good cost is efficiency.

Plasma deposition procedures involve the deposition of organic species from the plasma phase on a substrate. For instance by applying a (volatile) monomer as the gas phase an organic layer the structure of which resembles the corresponding polymer can be deposited. By applying a (volatile) monomer that possesses a chemical functionality a chemical functional polymeric layer can be obtained.

The plasma may be deposited from a monomer preferably being selected from the group consisting essentially of:
unsaturated monomers; acrylic acid, allyl amine, allyl isocyanate, allyl mercaptan, methacrylic acid, allyl alcohol, allyl acetate, allyl acetic acid, allyl glycidyl ether, 3 allyloxy, 1-2 propanediol, vinyl acetate, acrylic acid halides,
saturated monomers; alcohols such as methanol, ethanol propanol, acids such as propionic acid, acetic acid and the like, formaldehyde, propionic aldehyde, glutardialdehyde, aminoethane, aminoethanol, ethylene oxide, acetone methane, ethane, propane and the like, whereby the substrate is provided with the corresponding functionality.

Apart from the plasma deposition of saturated and unsaturated monomers, a functionality can be created in situ, i.e. in the plasma layer, by means of rearrangements of (cyclic) monomers or reaction between a mixture of plasma gases for example, whereafter this in-situ created functionality can be deposited.

Surfaces with a high surface energy, such as metal surfaces in general, may give rise to a rapid surface hydrophobisation due to contamination of the surface by species from its environment. This surface contamination may be disastrous for further surface modification for instance with respect to the stability of the final surface. Therefore this surface contamination should be prevented as much as possible by storing the surfaces in an inert atmosphere and reduction of the time between surface preparation and modification or the surface needs to be cleaned before modification. Plasma etching offers an excellent method for this cleaning. Plasma cleaning is fast and is a clean process in itself since it does not involve the use of organic solvent or substantial amounts of reagents that may have adverse effects on the environment. For the present invention it is advantageous to include an in situ plasma cleaning step of the substrate before the actual modification by plasma deposition.

The plasma deposited layer preferably comprises one or more sulphur compounds, for example thiols, sulfides and/or disulfides, i.e. in the form of mercaptoacetic acid, 2-mercaptopropionic acid, 3-mercaptopropionic acid, 1-mercaptopropenol, 2-mercaptoethanol and the like, preferably diallylsulfide, since, especially when gold is chosen as the substrate, an improved stability is provided.

According to a further aspect of the present invention there is provided a process for investigating he interaction of chemical and/or biological species, for example real time surface interactions, according to claims 14 or 15.

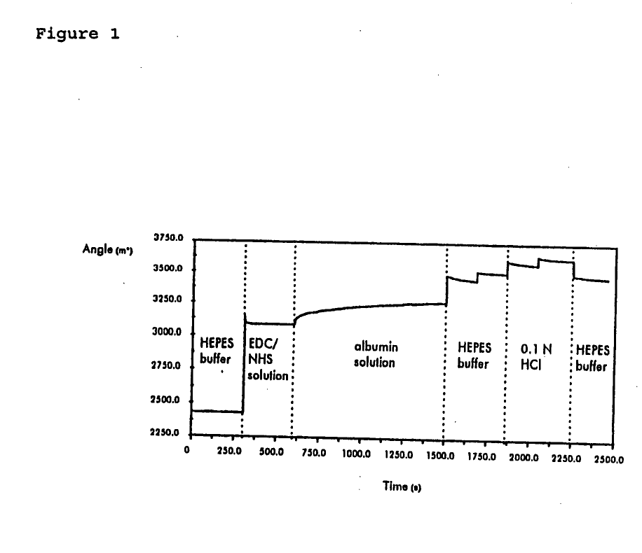

The invention will now be further clarified by way of the following examples, with reference to FIG. 1 which graphically shows the immobilization of albumins onto a COOH disk as carried out in example 12.

### EXAMPLE 1

### Preparation of Carboxylic Acid Functional Gold Surfaces

Gold coated glass discs (60) were placed in the central position of the plasma reactor which consisted of a glass tubes (l=150 cm, o=10 cm) with three electrodes positioned at the outside of the glass tube with the powered electrode in the center and two grounded electrodes positioned at 30 cm distance on both sides of the powered electrode. The electrodes were connected to an RF-generator (13.56 MHz, ENI ACG-3, ENI Power Systems) through a matching network (ENI Matchwork 5) and a matching network control unit (ENI TH-1000, ENI). The generator was controlled by a timer (Apple Ile computer with a time control program).

The reactor was evacuated to a pressure less than 0.001 mbar by a rotary pump (DUO 004 B, Pfeifer) which was equipped with a filter (ONF 025, Pfeifer) to prevent oil back streaming. The pressure was measured by a pressure gauge (Baratron 628A01MDE, MKS Instruments) and read from a display module (PR4000, MKS Instruments). An air flow of 5 sccm/min resulting in a pressure of about 0.12 mbar, was established for 5 minutes after which the discs were treated with a dynamic air plasma (85 W) for 1 minute at the same flow conditions. Air flow was controlled by a mass flow controller (type 1259+PR3000 control unit, MKS Instruments). After the plasma treatment the air flow was continued for 2 minutes and then stopped and an acrylic acid flow was established through the reactor via a direct monomer inlet resulting in a pressure of about 0.03 mbar. To prevent the acrylic acid to reach the pump after leaving the reactor, the acrylic acid flow was bypassed through a cold trap that as cooled with liquid nitrogen. The temperature of the acrylic acid in the storage container was room temperature. After two minutes the surfaces were treated with 5 pulses of an acrylic acid plasma at a discharge power of 75 (W), the pulses being separated from each other by 30 seconds of acrylic acid flow through the reactor. After the final pulse the surface were exposed to 2 additional minutes of acrylic acid flow whereupon the acrylic acid flow was stopped and the reactor was brought to atmospheric pressure with air.

### EXAMPLE 2

### Preparation of Amine Functional Surfaces

Gold coated glass discs (60) were placed in the plasma reactor as described in example 1. The reactor was evacuated to a pressure of less than 0.05 mbar and an air flow of 5 sccm/min was established for 5 minutes whereupon the discs were treated with a dynamic air plasma (85 W) for 1 minute at the same flow conditions. Then air flow was stopped and an allyl amine flow (0.07 mbar) was established through the reactor the temperature of the monomer storage container was 36° C. After two minutes the surfaces were treated with 10 pulses of an allyl amine plasma at a discharge power of 75 W separated from each other by 10 seconds of allyl amine flow through the reactor. After the final pulse the surfaces were exposed to 2 additional minutes of allyl amine flow after which the allyl amine flow was stopped and the reactor was brought to atmospheric pressure with air.

### EXAMPLE 3

Gold coated substrates (6) were placed in the plasma reactor (see example 2) between the cold electrode on the gas inlet side of the reactor and the hot electrode. The reactor was evacuated to a pressure less than 0.005 mbar and an air flow of 5 sccm was established through the reactor. After 2 minutes of air flow—the substrates were treated with a dynamic air plasma (5 sccm, 85 W) for 1 minute and subsequently exposed to an air flow of 5 sccm for 10 minutes again. Then the air flow was stopped and after evacuation of the reactor, an allylamine flow at a pressure of 0.095 mbar was established through the reactor. After two minutes allylamine flow the substrates were exposed to ten pulses of 1 second of an allylamine plasma at a discharge power of 85 W, the pulses being separated by ten seconds allylamine flow. After the final allylamine plasma pulse the allylamine flow was continued for 2 minutes after which the flow was discontinued, the reactor was evacuated and subsequently brought to atmospheric pressure with air. Following, the surfaces were analyzed for carbon, oxygen, nitrogen and gold by X-ray photo-electron spectroscopy, of which the results are shown in the table below. Also surfaces that were rinsed with water for 1 hr and subsequently dried were analyzed by XPS.

TABLE 1

surface composition (at %0)

| element | before rinsing | after rinsing |
|---|---|---|
| C | 65.4 | 62.4 |
| O | 10.3 | 10.5 |
| N | 17.5 | 13.6 |
| Au | 6.8 | 13.4 |

### EXAMPLE 4

Gold coated substrates (6) were placed in the plasma reactor (see example 2) between the cold electrode on the gas inlet side of the reactor and the hot electrode. The reactor was evacuated to a pressure of less than 0.005 mbar and an argon flow of 5 sccm was established through the reactor. After 2 minutes of argon flow the substrates were treated with a dynamic argon plasma (5 sccm, 85 W) for 1 minute and subsequently exposed to an argon flow of 5 sccm for 10 minutes again. Then the argon flow was stopped and after evacuation of the reactor, an allylamine flow at a pressure of 0.095 mbar was established through the reactor. After two minutes allylamine flow the substrates were exposed to ten pulses of 1 second of an allylamine plasma at a discharge power of 85 W, the pulses being separated by ten seconds allylamine flow. After the final allylamine plasma pulse the allylamine flow was continued for 2 minutes after which the flow was discontinued, the reactor was evacuated and subsequently brought to atmospheric pressure with air.

### EXAMPLE 5

Gold coated substrates (6) were placed in the plasma reactor (see example 2) between the cold electrode on the gas inlet side of the reactor and the hot electrode. The reactor was evacuated to a pressure of less than 0.005 mbar and an air flow of 5 sccm was established through the reactor. After 2 minutes of air flow the substrates were treated with a dynamic air plasma (5 sccm, 85 W) for 1 minute and subsequently exposed to an air flow of 5 sccm for 10 minutes again. Then the air flow was stopped and after evacuation of the reactor, an allylamine flow at a pressure of 0.095 mbar was established through the reactor. After two minutes allylamine flow the substrates were exposed to five pulses of 1 second of an allylamine plasma at a discharge power of 170 W, the pulses being separated by ten seconds allylamine flow, followed by five pulses of an allylamine plasma at a discharge power of 85 W, again the pulses being separated by ten seconds allylamine flow. After the final allylamine plasma pulse the allylamine flow was continued for 2 minutes after which the flow was discontinued, the reactor was evacuated and subsequently brought to atmospheric pressure with air. Following, the surfaces were analyzed for carbon, oxygen, nitrogen and gold by X-ray photo-electron spectroscopy, of which the results are shown in the table below.

TABLE 2

| | surface composition |
|---|---|
| element | (atomic %) |
| C | 62.8 |
| O | 9.8 |
| N | 20.8 |
| Au | 6.6 |

### EXAMPLE 6

Gold coated substrates (6) were placed in the plasma reactor (see example 2) between the cold electrode on the gas inlet side of the reactor and the hot electrode. The reactor was evacuated to a pressure of less than 0.005 mbar and an air flow of 5 sccm was established through the reactor. After 2 minutes of air flow the substrates were treated with a dynamic air plasma (5 sccm, 85 W) for 1 minute and subsequently exposed to an air flow of 5 sccm for 10 minutes again. Then the air flow was stopped and after evacuation of the reactor, a mixed flow of allylamine and octadiene (66 v % allylamine) at a pressure of 0.055 mbar was established through the reactor. After two minutes allylamine/octadiene flow the substrates were exposed to ten pulses of 1 second of an allylamine/octadiene plasma at a discharge power of 85 W, the pulses being separated by ten seconds allylamine/octadiene flow. After the final plasma pulse the allylamine/octadiene flow was continued for 2 minutes after which the flow was discontinued, the reactor was evacuated and subsequently brought to atmospheric pressure with air. Following, the surfaces were analyzed for carbon, oxygen, nitrogen and gold by X-ray photo-electron spectroscopy, of which the results are shown in the table below.

TABLE 3

| | surface composition |
|---|---|
| element | (atomic %) |
| C | 73.1 |
| O | 7.9 |
| N | 11.7 |
| Au | 7.3 |

### EXAMPLE 7

Gold coated substrates (6) were placed in the plasma reactor (see example 2) between the cold electrode on the gas inlet side of the reactor and the hot electrode. The reactor was evacuated to a pressure of less than 0.005 mbar and an air flow of 5 sccm was established through the reactor. After 2 minutes of air flow the substrates were treated with a dynamic air plasma (5 sccm, 85 W) for 1 minute and subsequently exposed to an air flow of 5 sccm for 10 minutes again. Then the air flow was stopped and after evacuation of the reactor, a mixed flow of allylamine and diallylsulfide (66 v % allylamine) at a pressure of 0.065 mbar was established through the reactor. After two minutes allylamine/diallylsulfide flow the substrates were exposed to ten pulses of 1 second of an allylamine/diallylsulfide plasma at a discharge power of 85 W, the pulses being separated by ten seconds allylamine/diallylsulfide flow. After the final plasma pulse the allylamine/diallylsulfide flow was continued for 2 minutes after which the flow was discontinued, the reactor was evacuated and subsequently brought to atmospheric pressure with air.

Following, the surfaces were analyzed for carbon, oxygen, nitrogen and gold by X-ray photo-electron spectroscopy, of which the results are shown in the table below. Also surfaces that were rinsed with water for 1 hr and subsequently dried were analyzed by XPS.

TABLE 4

surface composition (at %0)

| element | before rinsing | after rinsing |
|---|---|---|
| C | 73.4 | 68.3 |
| O | 4.4 | 5.3 |
| N | 8.3 | 9.0 |
| S | 13.3 | 16.5 |
| Au | 0.7 | 0.9 |

### EXAMPLE 8

Gold coated substrates (6) were placed in the plasma reactor (see example 2) between the cold electrode on the gas inlet side of the reactor and the hot electrode. The reactor was evacuated to a pressure of less than 0.005 mbar and an air flow of 5 sccm was established through the reactor. After 2 minutes of argon flow the substrates were treated with a dynamic argon plasma (5 sccm, 85 W) for 1 minute and subsequently exposed to an argon flow of 5 sccm for 10 minutes again. Then the argon flow was stopped and after evacuation of the reactor, a mixed flow of allylamine and diallylsulfide (66 v % allylamine) at a pressure of 0.065 mbar was established through the reactor. After two minutes allylamine/diallylsulfide flow the substrates were exposed to ten pulses of 1 second of an allylamine/diallylsulfide plasma at a discharge power of 85 W, the pulses being separated by ten seconds allylamine/diallylsulfide flow. After the final plasma pulse the allylamine/diallylsulfide flow was continued for 2 minutes after which the flow was discontinued, the reactor was evacuated and subsequently brought to atmospheric pressure with air.

### EXAMPLE 9

Gold coated substrates (6) were placed in the plasma reactor (see example 2) between the cold electrode on the gas inlet side of the reactor and the hot electrode. The reactor was evacuated to a pressure of less than 0.005 mbar and an air flow of 5 sccm was established through the reactor. After 2 minutes of air flow the substrates were treated with a dynamic air plasma (5 sccm, 85 W) for 1 minute and subsequently exposed to an air flow of 5 sccm for 10 minutes again. Then the air flow was stopped and after evacuation of the reactor to a pressure less than 0.005 mbar, a diallylsulfide flow at a pressure of 0.025 mbar was established through the reactor. After two minutes diallylsulfide flow the substrates were exposed to ten pulses of 1 second of an diallylsulfide plasma at a discharge power of 85 W, the pulses being separated from each other by ten seconds diallylsulfide flow. After the final diallylsulfide plasma pulse the diallylsulfide flow was continued for 1 minute after which the flow was discontinued and the reactor was evacuated to a pressure less than 0.001 mbar. Then an allylamine flow at a pressure of 0.090 mbar was established through the reactor. After two minutes allylamine flow the substrates were exposed to ten pulses of 1 second of an allylamine plasma at a discharge power of 85 W, the pulses being separated by ten seconds allylamine flow. After the final allylamine plasma pulse the allylamine flow was continued for 2 minutes whereafter the flow was discontinued and the reactor was evacuated to a pressure less than 0.001 mbar and brought to atmospheric pressure with air.

Following, the surfaces were analyzed for carbon, oxygen, nitrogen sulphur and gold by X-ray photo-electron spectroscopy, of which the results are shown in the table below. Also surfaces that were rinsed with water for 1 hr and subsequently dried were analyzed by XPS.

TABLE 5

surface composition (at %0)

| element | before rinsing | after rinsing |
|---|---|---|
| C | 69.8 | 68.3 |
| O | 6.9 | 10.2 |
| N | 14.8 | 12.9 |
| S | 8.5 | 8.6 |
| Au | 0.0 | 0.0 |

### EXAMPLE 10

Gold coated substrates (6) were placed in the plasma reactor (see example 2) between the cold electrode on the gas inlet side of the reactor and the hot electrode. The reactor was evacuated to a pressure of less than 0.005 mbar and an argon flow of 5 sccm was established through the reactor. After 2 minutes of argon flow the substrates were treated with a dynamic argon plasma (5 sccm, 85 W) for 1 minute and subsequently exposed to an argon flow of 5 sccm for 10 minutes again. Then the argon flow was stopped and after evacuation of the reactor to a pressure less than 0.005 mbar, a diallylsulfide flow at a pressure of 0.025 mbar was established through the reactor. After two minutes diallylsulfide flow the substrates were exposed to ten pulses of 1 second of an diallylsulfide plasma at a discharge power of 85 W, the pulses being separated from each other by ten seconds diallylsulfide flow. After the final diallylsulfide plasma pulse the diallylsulfide flow was continued for 1 minute after which the flow was discontinued and the reactor was evacuated to a pressure less than 0.001 mbar. Then an allylamine flow at a pressure of 0.090 mbar was established through the reactor. After two minutes allylamine flow the substrates were exposed to ten pulses of 1 second of an allylamine plasma at a discharge power of 85 W, the pulses being separated by ten seconds allylamine flow. After the final allylamine plasma pulse the allylamine flow was continued for 2 minutes after which the flow was discontinued and the reactor was evacuated to a pressure less than 0.001 mbar and brought to atmospheric pressure with air.

### EXAMPLE 11

### Coupling of CMD onto Amine Functionalized Gold Surfaces

Carboxymethyl cellulose (100 mg) was dissolved in 10 ml 0.05 M 2-(N-morpholino) ethanesulfonic acid after which 5 mg N-hydroxysuccinimid was added. After complete dissolution of this reagent 20 mg N-(3-dimethylaminopropyl)-N′ ethylcarbodiimide was added. After 3 minutes activation, an amine functionalized gold surface was incubated with 1 ml of this carboxymethyl dextran solution for 2,5 hours. Then the surfaces were rinsed with phosphate buffered saline, and water and vacuum dried. The whole immobilization procedure was performed at room temperature.

In this example, carboxymethyldextran is used as a model compound for chemical functional group containing compounds in general including but not limited to dextrans including carboxymethyl dextran, carboxymethyl cellulose, mono- di- oligo- and poly-saccharides, gum xanthan, carboxylate and amine dendrimers, and mono-, homo- and hetero-functional carboxylate polyethylene glycols and polyethylene oxide, polyethylene imine, polyacrylic acid, polyvinyl alcohol, etc.

The amount of these functional group containing compounds that is immobilized can be controlled by the reaction parameters such as reaction time, the concentration of the functional group containing compound and the ratio of coupling agent to functional group containing compound.

### EXAMPLE 12

### Immobilization of Albumin on a COOH-Functionalizes Sensing Device

A sensor device, that was COOH-functionalized by the plasma deposition method was used for the immobilization of albumin. During the immobilization procedure that was performed at 22.5° C. the surface events were monitored by Surface Plasmon Resonance Spectroscopy of which the results are given in FIG. 1. After mounting the functionalized sensing device in the SPR apparatus, the sensing surface was incubated with 10 mM HEPES buffer for about 5 minutes. Then the HEPES buffer was exchanged for a EDC (20 mg/ml) —NHS (4 mg/ml) solution in water. After 5 minutes activation the EDC/NHS solution was exchanged for an albumin solution (2 mg/ml in 10 mM HEPES) and an immobilization time of 15 minutes was applied. Then the sensing surface was rinsed with HEPES buffer and the stability of the immobilized albumin in HEPES buffer was monitored for 3 minutes after which the rinsing procedure with HEPES buffer was repeated. To study the stability of the immobilized albumin in 0.1 N HCl the HEPES buffer was replaced by 0.1 HCl and the sensing surface was incubated in this solution for 3 minutes after which 0.1 N HCl was replaced for fresh 0.1 N HCl and the measurement was continued for 3 minutes. Then the surface was rinsed with 0.1 N HEPES buffer again an incubation of the sensing surface was proceeded in this buffer for a final 5 minutes.

The results show that upon activation of the sensing surface with EDC/NHS and subsequent immobilization of albumin and rinsing with HEPES buffer the response increases with about 700 milli-degrees indicating the immobilization of albumin on the COOH-functionalized sensing surface. Rinsing of the surface with 0.1 N HCl only resulted in a decrease of the signal of about 30 milli-degrees, showing that the albumin immobilization is very stable.

The invention is not limited to the above description; the requested rights are rather determined by the following claims.

1-24. (canceled)

25. A device for investigating reactions between interactive chemical or biological species, said device comprising:
a substrate comprising a film of free electron metal consisting essentially of gold; and
an organic species deposited on the substrate from a plasma phase, thereby forming an organic plasma layer thereon, characterized in that prior to depositing the organic species one or more sulfur-containing compounds is mixed with the organic species in the plasma phase, wherein the presence of one or more sulfur-containing compounds enhances the stability of the plasma layer.

26. The device according to claim 25, wherein the plasma layer comprises one or more chemical or biological functional groups.

27. The device according to claim 26, further comprising one or more layer(s) deposited by means of wet chemically procedures, arranged on the plasma layer.

28. The device according to claim 25, wherein the one or more sulfur-containing compounds is selected from the group consisting of thiols, sulfides, disulfides and diallyl sulfide.

29. The device according to claim 28, wherein the substrate consists essentially of gold.

30. A process for producing a device for investigating reactions between interactive chemical and biological species, said process comprising the steps of (a) providing a pre-selected substrate, said substrate comprising a film of free electron metal consisting essentially of gold and (b) arranging an organic plasma layer comprising a mixture of an organic species and one or more sulfur-containing compounds in a plasma phase directly on the gold film by plasma deposition and defining a stable organic plasma layer.

31. The process according to claim 30, wherein the substrate consists essentially of gold, and wherein the plasma layer is directly deposited onto the substrate.

32. The process according to claim 30, wherein the organic species is derived from a monomer/oligomer/polymer in gas form, said monomer being saturated, partially saturated or unsaturated.

33. The process according to claim 30, wherein the organic species is selected from the group consisting essentially of acrylic acid, allyl amine, allyl isocyanate, allyl mercaptan, methacrylic acid, allyl alcohol, allyl acetate, allyl acetic acid, allyl glycidyl ether, 3 allyloxy, 1-2 propanediol, vinyl acetate, acrylic acid halides, methanol, ethanol, propanol, propionic acid, acetic acid, formaldehyde, propionic aldehyde, glutardialdehyde, aminoethane, aminoethanol, ethylene oxide, acetone, methane, ethane and propane.

34. The process according to claim 30, wherein the substrate is subjected to a pre-cleaning step comprising pre-treating the substrate by means of a plasma etching step before the plasma deposition step.

35. The process according to claim 30, wherein the gas plasma is deposited under the following conditions:
a discharge power of up to 5000 W;
an exposure duration of up to 1000 s;
a plasma gas flow of up to 10000 cm³/min;
a pressure of up to 1 bar; and
a frequency covering DC, AC, RF, and the MW ranges.

36. The process according to claim 35, wherein the gas plasma is deposited under the following conditions:
a discharge power of up to 500 W;
an exposure duration of up to 100 s;
a plasma gas flow of up to 100 cm³/min;
a pressure between 0.001-50 mbar; and
a frequency between 2-60 Mhz,
wherein the discharge power is pulsed to the plasma, the pulse discharges being separated by up to 100 s.

37. The process according to claim 35, wherein the substrate is treated in an acid flow.

38. The process according to claim 36, wherein following pulse discharge, the substrate is after-treated with a pre-selected gas.

39. The process for providing a device according to claim 30, suitable for investigate reactions between interactive bio/chemical species by means of surface plasmon resonance spectroscopy, said process comprising the steps of:
preselecting a free electron metal substrate, which metal substrate is suitable for allowing investigation by surface plasmon resonance spectroscopy, arranging a pre-selected first functional group species on the free electron metal substrate by means of plasma deposition, which first functional group species protects the free electron metal substrate from a second functional group species whose interaction with the plasma deposited first functional group species can be investigated, thereby preventing undesirable interactions between the free electron metal substrate and the second functional group species, and which first functional group species provides functionality for the second functional group species; and
subsequently arranging a second functional group species on the plasma deposited layer of the first functional group species, whereafter interaction between the first and second functional group species layers, can be investigated by means of surface plasmon resonance spectroscopy.

40. The process for providing a device according to claim 30, suitable for investigating reactions between interactive bio/chemical species by means of surface plasmon resonance spectroscopy, said process comprising the steps of;
preselecting a free electron metal substrate, which metal substrate is suitable for investigating and sensing surface interactions by surface plasmon resonance spectroscopy, arranging a pre-selected first functional group species on the free electron metal substrate by means of plasma deposition, which first functional group species protects the free electron metal substrate from a second functional group species whose interaction with the plasma deposited first functional group species can be investigated, thereby preventing undesirable interactions between the free electron metal substrate and the second functional group species, and which first functional group species provides functionality for the second functional group species.

41. The process according to claim 39, wherein before being exposed to the second functional group species, a bio/chemical functional layer is wet chemically arranged on the plasma deposited first functional group species layer, said wet chemically arranged functional layer being pre-selected for its specificity for the second functional group species and for the prevention of non specific interactions with the said second functional group species.

42. A method for investigating the interaction, of pre-determined chemical or biological species, comprising the step of analyzing the interaction between chemical or biological species arranged on a device according to claim 25.

43. A method for investigating the reaction between chemically interactive species, comprising the step of exposing or depositing chemical or biological species on the device of claim 25.

44. A method for investigating reactions between interactive bio/chemical species, by means of surface plasmon resonance spectroscopy, by the device of claim 25, wherein the method comprises the steps of pre-selecting a free electron metal substrate consisting essentially of gold, and a pre-selected organic plasma layer, wherein the organic plasma layer comprises a mixture of an organic species and one or more sulfur-containing compounds in a plasma phase, and arranging on the free electron metal substrate the pre-selected organic plasma layer, which plasma functional group species having both attachment ability to the free electron metal substrate, and specificity to further functional group species, whereby the interaction therebetween is investigatable by means of surface plasmon resonance spectroscopy.

45. The process according to claim 38, wherein the arranged organic plasma layer is derived from a compound having at least one functional group species, and wherein the gas comprises the at least one functional group species.
